(19) United States
(12) Patent Application Publication
(10) Pub. No.: US 2012/0171149 A1
(43) Pub. Date: Jul. 5, 2012

(54) AGENTS FOR TREATING KERATIN FIBERS, CONTAINING DIMETHYLSILANOL HYALURONATES AND GLYCEROL

(75) Inventors: Marlene Battermann, Asendorf (DE); Thomas Hippe, Appen (DE)

(73) Assignee: Henkel AG & Co. KGaA, Dusseldorf (DE)

(21) Appl. No.: 13/417,453

(22) Filed: Mar. 12, 2012

Related U.S. Application Data
(63) Continuation of application No. PCT/EP2010/063953, filed on Sep. 22, 2010.

(30) Foreign Application Priority Data
Sep. 24, 2009 (DE) 10 2009 044 948.5

(51) Int. Cl. A61K 8/73; A61K 8/97; A61Q 5/12

(52) U.S. Cl. 424/70.122; 132/202

(57) ABSTRACT

A hair treatment agent includes dimethylsilanol hyaluronates and glycerol.

### CROSS-REFERENCES TO RELATED APPLICATIONS

This application is a continuation of PCT/EP2010/063953, filed on Sep. 22, 2010, which claims priority under 35 U.S.C. §119 to DE 10 2009 044 948.5 filed on Sep. 24, 2009, both of which are hereby incorporated by reference.

### FIELD OF THE INVENTION

The present invention generally relates to hair treatment agents and to the use of said agents for treating hair.

### BACKGROUND OF THE INVENTION

The importance of care products that have as long-lasting effect as possible is increasingly growing, This is not least because of the heavy loading on the hair, resulting for example from coloring or permanent waving and from washing the hair with shampoos as well as from environmental pollution.

However, the known active ingredients cannot cover all requirements to an adequate extent. There is therefore still a demand for active ingredients or for combinations of active ingredients for cosmetic agents having good care properties. This applies in particular to the deep-down care of keratinic fibers, known as restructuring, inside the keratinic fibers.

Furthermore, other desirable features and characteristics of the present invention will become apparent from the subsequent detailed description of the invention and the appended claims, taken in conjunction with this background of the invention.

### BRIEF SUMMARY OF THE INVENTION

Surprisingly it has now been found that a composition containing dimethylsilanol hyaluronates and glycerol restructures the keratinic fibers inside the fibers, in other words provides deep-down care.

At the same time the use of these combinations leads to surprisingly good properties in the treated hair, in particular to improved combability and to an improved elasticity as well as to a marked increase in the washing resistance of colored hair and to a longer hold combined at the same time with a better shaping performance in waving processes such as finger waving and permanent waving. In particular, however, the gloss of the hair treated therewith is increased markedly.

These positive properties are obtained both with a typical rinse-off and with a leave-on application.

The present invention therefore firstly provides a composition for treating keratinic fibers, in particular human hair. The composition includes:

a) dimethylsilanol hyaluronates,

b) glycerol and

c) a cosmetic carrier.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Where reference is made below to the active ingredient complex (A), this relates to the ingredients mandatorily contained in the agents according to the invention.

Hair treatment agents within the meaning of the present invention are, for example, hair conditioners, hair sprays, hair rinses, hair masks, hair packs, hair tonics, hair fixing agents, hair setting agents, hair styling preparations, blow-drying lotions, styling mousses, hair gels, hair waxes or combinations thereof. Preferred agents according to the invention are shampoos, conditioning agents or hair tonics.

According to the invention combability is understood to mean both the combability of wet fibers and the combability of dry fibers. The combing work applied or the force applied during combing of a group of fibers serves as a measure of combability. The measurement parameters can be assessed by sensory means by the person skilled in the art or quantified by means of measuring devices.

Feel is defined as the tactility of a group of fibers, the person skilled in the art feeling and assessing the parameters fullness and softness of the group of fibers by sensory means.

Dimethylsilanol hyaluronate is the INCI name for a product consisting of hyaluronic acid and dimethyl silanol. This raw material is already successfully used in skin cosmetics. The teaching according to the invention naturally also encompasses the use of the alkali metal salts of dimethylsilanol hyaluronate. Hyaluronic acid is itself a constituent of the natural moisture-retaining factor of the skin. Dimethylsilanol hyaluronate is used in the compositions according to the invention in an amount from 0.001 to 5 wt. %. Amounts from 0.01 to 5.0 wt. % are preferred, and most highly preferably from 0.01 to 2.5 wt. %.

Glycerol has already long been used in numerous cosmetic compositions and preferably as a moisturizing raw material. Conventionally, however, glycerol is used in relatively large amounts of several percent by weight. Relatively large amounts of glycerol have the disadvantage, however, that they place a load on keratinic fibers in particular, so that they are no longer easy to style. The appearance of keratinic fibers treated with glycerol is perceived as wet and unpleasant. However, the active ingredient combination according to the invention uses glycerol in only small amounts. Compositions according to the invention contain glycerol in an amount from 0.01 to 5 wt. %, preferably from 0.05 wt. % to 2.5 wt. %, more preferably from 0.05 wt. % to 1.5 wt. % and most highly preferably in amounts from 0.1 to 1.0 wt. %.

O/W, W/O and W/O/W emulsions in the form of creams or gels, or other preparations that are suitable in particular for use on the hair, are suitable in particular according to the invention as cosmetic carriers c). The cosmetic carriers can in particular be aqueous or aqueous-alcoholic.

An aqueous cosmetic carrier contains at least 50 wt. % of water.

Within the meaning of the present invention aqueous-alcoholic cosmetic carriers are understood to be aqueous solutions containing 3 to 70 wt. % of a C₁-C₆ alcohol, in particular methanol, ethanol or propanol, isopropanol, butanol, isobutanol, tert-butanol, n-pentanol, isopentanols, n-hexanol, isohexanols, glycol, 1,2-pentanediol, 1,5-pentanediol, 1,2-hexanediol or 1,6-hexanediol. The agents according to the invention can additionally contain further organic solvents, such as for example methoxybutanol, benzyl alcohol, ethyl diglycol or 1,2-propylene glycol. All water-soluble organic solvents are preferred here.

The present active ingredient combination according to the invention is preferably presented as a rinse-off application or as a leave-on application. Within the leave-on applications, presentation as a non-aerosol spray is particularly preferred. Rinse-off applications are preferably hair masks, shampoos and hair conditioning agents.

Surfactants are used as further ingredients, with both anionic and zwitterionic, ampholytic, non-ionic and cationic surfactants being suitable in principle. The choice is governed by the type of agent. In the case of a shampoo, at least one surfactant from the group of anionic, zwitterionic or non-ionic surface-active substances is chosen in particular. It is preferable here for at least one anionic and at least one zwitterionic surface-active substance to be chosen. These surface-active substances are particularly preferably chosen from the group of particularly gentle surface-active substances. The ratio between anionic and zwitterionic surface-active substances is between 10:1 and 1:5. The ratio is particularly preferably 5:1 to 1:2.

All anionic surface-active substances that are suitable for use on the human body are suitable as anionic surfactants (Tanion) in preparations according to the invention. Typical examples of anionic surfactants are:
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16, and salts thereof
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms, alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
hydroxyl sulfonates substantially corresponding to at least one of the following two formulae or mixtures thereof and salts thereof, CH₃—(CH₂)_{y}—CHOH—(CH₂)ₚ—(CH—SO₃M)-(CH₂)_{z}—CH₂—O—(CₙH₂ₙO)ₓ—H, and/or CH₃—(CH₂)_{y}—(CH—SO₃M)-(CH₂)ₚ—CHOH—(CH₂)_{z}—CH₂—O—(CₙH₂ₙO)ₓ—H, wherein in both formulae y and z=0 or whole numbers from 1 to 18, p=0, 1 or 2 and the sum (y+z+p) is a number from 12 to 18, x=0 or a number from 1 to 30 and n is a whole number from 2 to 4, and M=H or alkali, in particular sodium, potassium, lithium, alkaline-earth, in particular magnesium, calcium, zinc, and/or an ammonium ion, which can optionally be substituted, in particular mono-, di-, tri- or tetraammonium ions having C1 to C4 alkyl, alkenyl or aryl residues,
sulfated hydroxyalkyl polyethylene and/or hydroxyalkylene propylene glycol ethers of the formula R¹—(CHOSO₃M)-CHR³—(OCHR⁴—CH₂)ₙ—OR² in which R¹ denotes a linear alkyl residue having 1 to 24 C atoms, R² denotes a linear or branched, saturated alkyl residue having 1 to 24 C atoms, R³ denotes hydrogen or a linear alkyl residue having 1 to 24 C atoms, R⁴ denotes hydrogen or a methyl residue and M denotes hydrogen, ammonium, alkyl ammonium, alkanol ammonium, wherein the alkyl and alkanol residues each have 1 to 4 C atoms, or a metal atom selected from lithium, sodium, potassium, calcium or magnesium, and n denotes a number in the range from 0 to 12 and furthermore the total number of C atoms contained in R¹ and R³ is 2 to 44,
sulfonates of unsaturated fatty acids having 8 to 24 C atoms and 1 to 6 double bonds,
esters of tartaric acid and citric acid with alcohols that are addition products of around 2 to 15 molecules of ethylene oxide and/or propylene oxide with fatty alcohols having 8 to 22 C atoms,
alkyl and/or alkenyl ether phosphates of the formula, R¹(OCH₂CH₂)ₙ—O—(PO—OX)—OR²,
in which R¹ preferably denotes an aliphatic hydrocarbon residue having 8 to 30 carbon atoms, R² denotes hydrogen, a (CH₂CH₂O)ₙR² residue or X, n denotes numbers from 1 to 10 and X denotes hydrogen, an alkali or alkaline-earth metal or NR³R⁴R⁵R⁶, with R³ to R⁶ independently of one another denoting hydrogen or a C₁ to C₄ hydrocarbon residue,
sulfated fatty acid alkylene glycol esters of the formula RCO(AlkO)ₙSO₃M in which RCO— denotes a linear or branched, aliphatic, saturated and/or unsaturated acyl residue having 6 to 22 C atoms, Alk denotes CH₂CH₂, CHCH₃CH₂ and/or CH₂CHCH₃, n denotes numbers from 0.5 to 5 and M denotes a metal, such as alkali metal, in particular sodium, potassium, lithium, alkaline-earth metal, in particular magnesium, calcium, zinc, or ammonium ion, such as ⁺NR³R⁴R⁵R⁶, with R³ to R⁶ independently of one another denoting hydrogen or a C₁ to C₄ hydrocarbon residue,
monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)_{z}—SO₃X, in which R⁸CO denotes a linear or branched acyl residue having 6 to 22 carbon atoms, x, y and z in total denote 0 or numbers from 1 to 30, preferably 2 to 10, and X denotes an alkali or alkaline-earth metal. Typical examples of suitable monoglyceride (ether) sulfates within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as the ethylene oxide adducts thereof with sulfur trioxide or chlorosulfonic acid in the form of their sodium salts. Monoglyceride sulfates are preferably used in which R⁸CO denotes a linear acyl residue having 8 to 18 carbon atoms,
amide ether carboxylic acids, R¹—CO—NR²—CH₂CH₂—O—(CH₂CH₂O)ₙCH₂COOM, with R¹ as a straight-chain or branched alkyl or alkenyl residue having a number of carbon atoms in the chain from 2 to 30, n denotes a whole number from 1 to 20 and R² denotes hydrogen, a methyl, ethyl, propyl, isopropyl, n-butyl, t-butyl or isobutyl residue and M denotes hydrogen or a metal such as alkali metal, in particular sodium, potassium, lithium, alkaline-earth metal, in particular magnesium, calcium, zinc, or an ammonium ion, such as ⁺NR³R⁴R⁵R⁶, with R³ to R⁶ independently of one another denoting hydrogen or a C₁ to C₄ hydrocarbon residue. Such products are available for example from Chem-Y under the product name Akypo®,
acyl glutamates of the formula XOOC—CH₂CH₂CH(C(NH)OR)—COOX, in which RCO denotes a linear or branched acyl residue having 6 to 22 carbon atoms and 0 and/or 1, 2 or 3 double bonds and X denotes hydrogen, an alkali and/or alkaline-earth metal, ammonium, alkyl ammonium, alkanol ammonium or glucammonium,
condensation products of a water-soluble salt of a water-soluble protein hydrolysate with a C₈-C₃₀ fatty acid. Such products have long been available commercially under the trademark Lamepon®, Maypon®, Gluadin®, Hostapon® KCG or Amisoft®,
alkyl and/or alkenyl oligoglycoside carboxylates, sulfates, phosphates and/or isethionates,
acyl lactylates and
hydroxy mixed ether sulfates.

If the gentle anionic surfactants contain polyglycol ether chains, it is most particularly preferable for them to have a narrow homolog distribution. In the case of gentle anionic surfactants having polyglycol ether units, it is furthermore preferable for the number of glycol ether groups to be 1 to 20, preferably 2 to 15, particularly preferably 2 to 12. Particularly gentle anionic surfactants having polyglycol ether groups without a narrow homolog distribution can also be obtained for example if the number of polyglycol ether groups is 4 to 12 and Zn or Mg ions are chosen as the counterion. One example is the commercial product Texapon® ASV.

Particularly suitable zwitterionic surfactants are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytic surfactants (Tampho) are understood to be surface-active compounds that are capable of forming internal salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Typical examples of amphoteric or zwitterionic surfactants are alkyl betaines, alkyl amido betaines, aminopropionates, aminoglycinates, imidazolinium betaines and sulfobetaines.

Particularly preferred ampholytic surfactants are N-cocoalkyl aminopropionate, cocoacylaminoethyl aminopropionate and C₁₂-C₁₈ acyl sarcosine.

Non-ionic surfactants (Tnio) are for example
addition products of 2 to 50 mol of ethylene oxide and/or 0 to 5 mol of propylene oxide with linear and branched fatty alcohols having 6 to 30 C atoms, fatty alcohol polyglycol ethers or fatty alcohol polypropylene glycol ethers or mixed fatty alcohol polyethers,
addition products of 2 to 50 mol of ethylene oxide and/or 0 to 5 mol of propylene oxide with linear and branched fatty acids having 6 to 30 C atoms, fatty acid polyglycol ethers or fatty acid polypropylene glycol ethers or mixed fatty acid polyethers,
addition products of 2 to 50 mol of ethylene oxide and/or 0 to 5 mol of propylene oxide with linear and branched alkylphenols having 8 to 15 C atoms in the alkyl group, alkylphenol polyglycol ethers or alkyl polypropylene glycol ethers or mixed alkylphenol polyethers,
addition products of 2 to 50 mol of ethylene oxide and/or 0 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydrol® LS, Dehydrol® LT (Cognis),
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as for example the commercial product Hydagen® HSP (Cognis) or Sovermol® types (Cognis),
alkoxylated triglycerides,
alkoxylated fatty acid alkyl esters of the formula (Tnio-1)

R¹CO—(CH₂CHR²)_{w}OR³ (Tnio-1)
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,
amine oxides,
hydroxy mixed ethers, R¹O[CH₂CH(CH₃)O]ₓ(CH₂CHR²O)_{y}[CH₂CH(OH)R³]_{z} with R¹ denoting a linear or branched, saturated or unsaturated alkyl and/or alkenyl residue having 2 to 30 C atoms, R² denoting hydrogen, a methyl, ethyl, propyl or isopropyl residue, R³ denoting a linear or branched alkyl residue having 2 to 30 C atoms, x denoting 0 or a number from 1 to 20, Y a number from 1 to 30 and z denoting the number 1, 2, 3, 4 or 5,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanol amides and fatty amines,
sugar surfactants of the alkyl and alkenyl oligoglycoside type,
sugar surfactants of the fatty acid-N-alkyl polyhydroxyalkylamide type,
fatty acid amide polyglycol ethers, fatty amine polyglycol ethers,
mixed ethers or mixed formals and polysorbates.

Furthermore, at least one quaternary imidazoline compound, i.e. a compound having a positively charged imidazoline ring, is preferably included as a cationic surfactant. Formula I below shows the structure of these compounds.

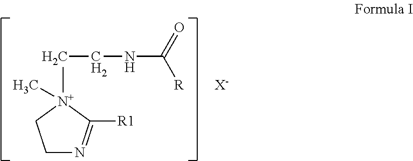

Residues R and R¹ independently of each other denote a saturated or unsaturated, linear or branched hydrocarbon residue having a chain length of 8 to 30 carbon atoms. The preferred compounds of formula I each contain the same hydrocarbon residue for R and R1. The chain length of residues R and R1 is preferably 12 carbon atoms. Compounds having a chain length of at least 16 carbon atoms and most particularly preferably at least 20 carbon atoms are particularly preferred. A most highly preferred compound of formula I has a chain length of 21 carbon atoms. A product of this chain length is known for example under the name Quaternium-91 or the trade names Crodazosoft® DBQ, which in addition to Quatemium-91 also contains Cetrimonium Methosulfate and Cetearyl Alcohol, and Crodazosoft® SCQ, which in addition to Quaternium-91 also contains PPG-3 Benzyl Ether Myristate. All physiologically tolerable counterions, for example methosulfates, halides such as chloride, fluoride, bromide, and also phosphates, are suitable as counterions. Particularly suitable examples according to the invention are available for example under the INCI names Quaternium-27, Quaternium-72, Quaternium-83 and Quaternium-91. The commercial products Crodazosoft® DBQ and Crodazosoft® SCQ, or Quaternium-91, are most preferably used.

The imidazolines of formula I are contained in the compositions according to the invention in amounts from 0.01 to 20 wt. %, preferably in amounts from 0.05 to 10 wt. % and most particularly preferably in amounts from 0.1 to 7.5 wt. %. The very best results are obtained with amounts from 0.1 to 5 wt. %, relative in each case to the complete composition of the individual agent.

The following cationic surfactants in accordance with formula (Tkat-2) can moreover be used.

RCO—X—N⁺R¹R²R³A⁻ (Tkat-2)

R here denotes a substituted or unsubstituted, branched or straight-chain alkyl or alkenyl residue having 11 to 35 carbon atoms in the chain,
X denotes —O— or —NR⁵—,
R¹ denotes an alkylene group having 2 to 6 C atoms, which can be non-substituted or substituted, wherein if it is substituted, substitution with an —OH or —NH group is preferred,
R², R³ independently of each other denote an alkyl or hydroxyalkyl group having 1 to 6 C atoms in the chain, wherein the chain can be straight or branched,
R⁵ denotes hydrogen or a C₁ to C₆ straight-chain or branched alkyl or alkenyl residue, which can also be substituted with a hydroxyl group.

Within this structural class, compounds of one of the following structures are preferably used:

CH₃(CH₂)₂₀CONH(CH₂)₃—N⁺(CH₃)₂—CH₂CH₃A⁻ (Tkat-3)

CH₃(CH₂)₂₀CONH(CH₂)₃—N⁺(CH₃)₂—CH₂(CHOH)CH₂OHA⁻ (Tkat-4)

CH₃(CH₂)₂₀COOCH₂CHOHCH₂—N⁺(CH₃)₃A⁻ (Tkat-5)

CH₃(CH₂)₂₀CONH(CH₂)₃—N⁺(CH₃)₂—CH₂CH₂OHA⁻ (Tkat-6)

Examples of such commercial products are Schercoquat BAS, Lexquat AMG-BEO, Akypoquat 131 or Incroquat Behenyl HE.

Esterquats according to the formula (Tkat1-2) can also be used.

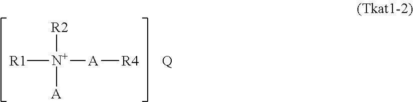

Here residues R1, R2 and R3 are independent of one another and can be identical or different. Residues R1, R2 and R3 denote:
a branched or unbranched alkyl residue having 1 to 4 carbon atoms, which can contain at least one hydroxyl group, or
a saturated or unsaturated, branched or unbranched or a cyclic saturated or unsaturated alkyl residue having 6 to 30 carbon atoms, which can contain at least one hydroxyl group, or
an aryl or alkaryl residue, for example phenyl or benzyl,
the residue (-A-R4), with the proviso that at most 2 of residues R1, R2 or R3 can denote this residue.

The residue -(A-R4) is included at least 1 to 3 times.

Here A denotes:
1) —(CH2)n- where n=1 to 20, preferably n=1 to 10 and particularly preferably n=1-5, or
2) —(CH2-CHR5-O)n- where n=1 to 200, preferably 1 to 100, particularly preferably 1 to 50, and particularly preferably 1 to 20 with R5 denoting hydrogen, methyl or ethyl, and R4 denotes:
1) R6-O—CO—, in which R6 is a saturated or unsaturated, branched or unbranched or a cyclic saturated or unsaturated alkyl residue having 6 to 30 carbon atoms, which can contain at least one hydroxyl group and which can optionally also be ethoxylated with 1 to 100 ethylene oxide units and/or 1 to 100 propylene oxide units, or
2) R7-CO—, in which R7 is a saturated or unsaturated, branched or unbranched or a cyclic saturated or unsaturated alkyl residue having 6 to 30 carbon atoms, which can contain at least one hydroxyl group and which can optionally also be ethoxylated with 1 to 100 ethylene oxide units and/or 1 to 100 propylene oxide units,
   and Q denotes a physiologically tolerable organic or inorganic anion.

Such products are sold under the trademarks Rewoquat®, Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such esterquats.

Other compounds of formula (Tkat1-2) that are particularly preferred according to the invention are included in formula (Tkat1-2.1), cationic betaine esters.

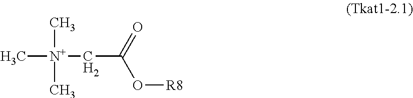

R8 corresponds in its meaning to R7.

Monoalkyl trimethylammonium salts having an alkyl residue chain length of 16 to 24 carbon atoms can be included as a further ingredient.

These compounds have the structure shown in formula (Tkat1-1)

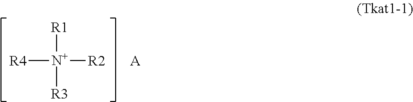

in which R1, R2 and R3 each denote a methyl group and R4 denotes a saturated, branched or unbranched alkyl residue having a chain length of 16 to 24 carbon atoms.

Examples of compounds of formula (Tkat1-1) are cetyl trimethylammonium chloride, cetyl trimethylammonium bromide, cetyl trimethylammonium methosulfate, stearyl trimethylammonium chloride, behenyl trimethylammonium chloride, behenyl trimethylammonium bromide and behenyl trimethylammonium methosulfate.

In a particularly preferred embodiment of the invention the agents according to the invention contain furthermore at least one amine and/or cationized amine, in particular an amidoamine and/or a cationized amidoamine of the following structural formulae:

R¹—NH—(CH₂)ₙ—NR²R³ (Tkat7) and/or

R¹—NH—(CH₂)ₙ—NR²R³R⁴ (Tkat8)

in which R1 denotes an acyl or alkyl residue having 6 to 30 C atoms, which can be branched or unbranched, saturated or unsaturated, and wherein the acyl residue and/or alkyl residue can contain at least one OH group, and
R2, R3 and R4 independently of one another denote hydrogen or an alkyl residue having 1 to 4 C atoms, which can be identical or different, saturated or unsaturated, and
X⁻ denotes an anion and
n denotes a whole number between 1 and 10.

A composition in which the amine and/or the quaternized amine according to the general formulae (Tkat7) and/or (Tkat8) is an amidoamine and/or a quaternized amidoamine is preferred, in which R1 denotes a branched or unbranched, saturated or unsaturated acyl residue having 6 to 30 C atoms, which can contain at least one OH group. A fatty acid residue from oils and waxes, in particular from natural oils and waxes, is preferred here. Suitable examples include lanolin, beeswax or candelilla wax.

Also preferred are amidoamines and/or quaternized amidoamines in which R2, R3 and/or R4 in formulae (Tkat7) and/or (Tkat8) denote a residue according to the general formula CH₂CH₂OR5, in which R5 can have the meaning of alkyl residues having 1 to 4 carbon atoms, hydroxyethyl or hydrogen. The preferred value of n in the general formulae (Tkat7) and/or (Tkat8) is a whole number between 2 and 5.

Also preferred are amidoamines and/or quaternized amidoamines of the general formulae (Tkat7) and/or (Tkat8) in which the anion X⁻ is a halide ion or a compound of the general formula RSO₃⁻, in which R has the meaning of saturated or unsaturated alkyl residues having 1 to 4 carbon atoms.

The alkyl residue having 1 to 4 carbon atoms of R2, R3 and R4 and/or the alkyl residue having 1 to 4 carbon atoms of RSO3⁻ in the general formula (Tkat7) and/or (Tkat8) can contain at least one hydroxyl group.

The alkyl amidoamines can be present as is and can also be converted into a quaternary compound in the composition by protonation in a correspondingly acid solution. Cationic alkyl amidoamines are preferred according to the invention.

Suitable amidoamines for use according to the invention, which can optionally be quaternized, include for example: Witcamine 100 (Witco, INCI name; Cocamidopropyl Dimethylamine), Incromine BB (Croda, INCI name: Behenamidopropyl Dimethylamine), Mackine 401 (McIntyre, INCI name. Isostearylamidopropyl Dimethylamine) and other Mackine types, Adogen S18V (Witco, INCI name: Stearylamidopropyl Dimethylamine), and as permanently cationic aminoamines: Rewoquat RTM 50 (Witco Surfactants GmbH, INCI name: Ricinoleamidopropyltrimonium Methosulfate), Empigen CSC (Albright & Wilson, INCI name: Cocamidopropyltrimonium Chloride), Swanol Lanoquat DES-50 (Nikko, INCI name: Quaternium-33), Rewoquat UTM 50 (Witco Surfactants GmbH, Undecyleneamidopropyltrimonium Methosulfate).

The anion of all cationic compounds listed above is selected from the physiologically tolerable anions. The halide ions, fluoride, chloride, bromide, sulfate of the general formula RSO₃⁻, in which R has the meaning of saturated or unsaturated alkyl residues having 1 to 4 carbon atoms, or anionic residues of organic acids such as maleate, fumarate, oxalate, tartrate, citrate, lactate or acetate, can be cited by way of example.

The aforementioned cationic surfactants can be used individually or in any combinations with one another, wherein they are included in amounts of between 0.01 to 20 wt. %, preferably in amounts from 0.01 to 10 wt. % and most particularly preferably in amounts from 0.1 to 7.5 wt. %. The very best results are obtained with amounts from 0.1 to 5 wt. %, relative in each case to the complete composition of the individual agent.

The surfactants (T) are used in amounts from 0.05 to 45 wt. %, preferably 0.1 to 30 wt. % and most particularly preferably from 0.5 to 25 wt. %, relative to the complete agent used according to the invention.

Most highly preferred compositions containing the active ingredient combination (A) have the characterizing feature that furthermore they contain at least one native oil or at least one ester oil or at least one silicone oil or at least one polymer.

Ester oils are understood to be the esters of C₆-C₃₀ fatty acids with C₂-C₃₀ fatty alcohols. The monoesters of fatty acids with alcohols having 2 to 24 C atoms are preferred. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate) (Cetiol®, lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

The ester oils can naturally also be alkoxylated with ethylene oxide, propylene oxide or mixtures of ethylene oxide and propylene oxide. The alkoxylation can take place both on the fatty alcohol part and on the fatty acid part and also on both parts of the ester oils. It is preferred according to the invention, however, for the fatty alcohol to be alkoxylated first and then esterified with fatty acid. These compounds are shown in general in the formula (D4-II).

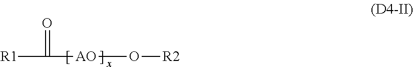

R1 here denotes a saturated or unsaturated, branched or unbranched, cyclic saturated, cyclic unsaturated acyl residue having 6 to 30 carbon atoms,
AO denotes ethylene oxide, propylene oxide or butylene oxide,
X denotes a number between 1 and 200, preferably between 1 and 100, particularly preferably between 1 and 50, most particularly preferably between 1 and 20, most highly preferably between 1 and 10 and most preferably between 1 and 5,

R2 denotes a saturated or unsaturated, branched or unbranched, cyclic saturated, cyclic unsaturated alkyl, alkenyl, alkynyl, phenyl or benzyl residue having 6 to 30 carbon atoms. Examples of fatty acid components used as residue R1 in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof. Examples of the fatty alcohol components as residue R2 in the ester oils are benzyl alcohol, isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. An ester oil that is particularly preferred according to the invention is available for example under the INCI name PPG-3 Benzyl Ether Myristate.

Ester oils are also understood to include:
dicarboxylic acid esters such as di-n-butyl adipate, di-(2-ethylhexyl)adipate, di-(2-ethylhexyl)succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate, and
symmetrical, asymmetrical or cyclic esters of carbonic acid with fatty alcohols, for example glycerol carbonate or dicaprylyl carbonate (Cetiol® CC), tri-fatty acid esters of saturated and/or unsaturated linear and/or branched fatty acids with glycerol,
fatty acid partial glycerides, namely monoglycerides, diglycerides and technical mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof. Oleic acid monoglycerides are preferably used.

The ester oils are used in the agents according to the invention in an amount from 0.01 to 20 wt. %, preferably 0.01 to 10.0 wt. %, particularly preferably 0.01 to 7.5 wt. %, most highly preferably from 0.1 to 5.0 wt. %. Naturally it is also possible according to the invention to use a plurality of ester oils at the same time.

Natural oils can furthermore additionally be used with the active ingredient combination (A). The natural oils can also be waxy or solid at room temperature. These oils, oil bodies, preferably have a melting point below 50° C., particularly preferably below 45° C., most particularly preferably below 40° C., most highly preferably below 35° C. and most preferably the cosmetic oils are free-flowing at a temperature below 30° C. According to the invention synthetic oils such as hydrocarbons are also understood to be natural oils. These oils are defined and described in more detail below.

The oils according to the invention include, for example:
vegetable oils. Examples of such oils are sunflower oil, olive oil, soya oil, rapeseed oil, almond oil, jojoba oil, orange oil, wheat germ oil, peach kernel oil and the liquid components of coconut butter. Other triglyceride oils are also suitable, however, such as the liquid components of beef fat and synthetic triglyceride oils
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds available as commercial products, 1,3-di-(2-ethylhexyl)cyclohexane (Cetiol® S) and di-n-octyl ether (Cetiol® OE), can be preferred.

Suitable natural oils according to the invention are for example amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil.

The natural oils are used in the agents according to the invention in an amount from 0.01 to 20 wt. %, preferably 0.01 to 10.0 wt. % particularly preferably 0.01 to 7.5 wt. %, most highly preferably from 0.1 to 5.0 wt. %. Naturally it is also possible according to the invention to use a plurality of the natural oils at the same time.

As a further most highly preferred ingredient to increase the effect of the active ingredient complex (A) the agents according to the invention preferably contain at least one silicone polymer selected from the group of dimethiconols and/or the group of amino-functional silicones and/or the group of dimethicones and/or the group of cyclomethicones.

The dimethicones according to the invention can be both linear and branched and also cyclic or cyclic and branched. Linear dimethicones can be represented by the following structural formula (Si1):

(SiR¹₃)—O—(SiR²₂—O—)ₓ—(SiR¹₃) (Si1)

Branched dimethicones can be represented by the structural formula (Si1.1):

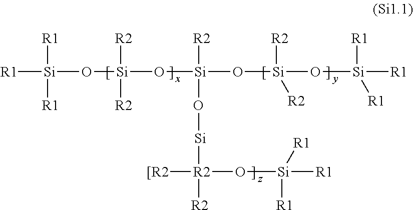

Residues R¹ and R² independently of each other denote hydrogen, a methyl residue, a C2 to C30 linear, saturated or unsaturated hydrocarbon residue, a phenyl residue and/or an aryl residue. The numbers x, y and z are whole numbers and each run independently of one another from 0 to 50,000. The molecular weights of the dimethicones are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs. Viscosities around the range of approximately 60,000 cPs are most highly preferred. Reference is made here by way of example to the product “Dow Corning 200 with 60,000 cSt”.

Particularly preferred cosmetic or dermatological preparations according to the invention have the characterizing feature that they contain at least one silicone of formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are included in the compositions according to the invention in amounts from 0.01 to 10 wt. %, preferably 0.01 to 8 wt. %, particularly preferably 0.1 to 7.5 wt. % and in particular 0.1 to 5 wt. %, relative to the complete composition.

Particularly preferred agents according to the invention contain one or more amino-functional silicones. Such silicones can be described for example by the formula (Si-2)

M(RₐQ_{b}SiO_{(4-a-b)/2})ₓ(R_{c}SiO_{(4-c)/2})_{y}M (Si-2)

in which in the above formula
R is a hydrocarbon or a hydrocarbon residue having 1 to approximately 6 carbon atoms,
Q is a polar residue of the general formula —R¹HZ,
   in which
   R¹ is a divalent linking group that is bonded to hydrogen and the residue Z, composed of carbon and hydrogen atoms, carbon, hydrogen and oxygen atoms or carbon, hydrogen and nitrogen atoms, and
   Z is an organic, amino-functional residue containing at least one amino-functional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2000, preferably from approximately 3 to approximately 50 and most preferably from approximately 3 to approximately 25, and
y is a number in the range from approximately 20 to approximately 10,000, preferably from approximately 125 to approximately 10,000 and most preferably from approximately 150 to approximately 1000, and
M is a suitable silicone end group as is known in the prior art, preferably trimethylsiloxy.

According to formula (Si-2) Z is an organic, amino-functional residue containing at least one functional amino group. A possible formula for said Z is NH(CH₂)_{z}NH₂, in which z is a whole number greater than or equal to 1. Another possible formula for said Z is —NH(CH₂)_{z}(CH₂)_{zz}NH, in which both z and zz are independently of each other a whole number greater than or equal to 1, this structure encompassing diamino ring structures, such as piperazinyl. Said Z is most preferably an —NHCH₂CH 2NH₂ residue. Another possible formula for said Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0.

Q according to formula (Si-2) is most preferably a polar amino-functional residue of the formula —CH₂CH₂CH₂NHCH₂CH₂NH₂.

In the formula (Si-2) a assumes values in the range from 0 to 2, b assumes values in the range from 2 to 3, a+b is less than or equal to 3, and c is a number in the range from 1 to 3. Cationic silicone oils such as the commercially available products Dow Corning (DC) 929 Emulsion, DC 2-2078, DC 5-7113, SM-2059 (General Electric) and SLM-55067 (Wacker) are suitable according to the invention.

Particularly preferred agents according to the invention have the characterizing feature that they contain at least one amino-functional silicone of the formula (Si-3a)

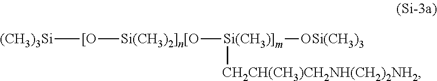

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10.

Under the INCI declaration these silicones are known as trimethylsilyl amodimethicone and they are available for example under the name Q2-7224 (manufacturer: Dow Corning; a stabilized trimethylsilyl amodimethicone).

Agents according to the invention are also particularly preferred that contain an amino-functional silicone of the formula (Si-3b)

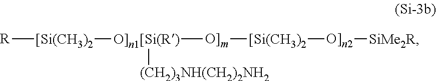

in which
R denotes —OH, an (optionally ethoxylated and/or propoxylated) (C₁ to C₂₀) alkoxy group or a —CH₃ group,
R′ denotes —OH, a (C₁ to C₂₀) alkoxy group or a —CH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Under the INCI declaration these silicones are known as amodimethicone or as functionalized amodimethicone, such as for example Bis(C13-15 Alkoxy) PG Amodimethicone (available for example as the commercial product DC 8500 from Dow Corning), Trideceth-9 PG-Amodimethicone (available for example as the commercial product Silcare Silicone SEA from Clariant). Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

in which residues R1 to R6 independently of each other denote C1 to C22 alkyl residues, which can contain hydroxyl groups, and wherein preferably at least one of the residues has at least 8 C atoms and the other residues have 1 to 4 C atoms,
residues R⁷ to R¹² independently of one another are identical or different and denote C₁ to C10 alkyl or phenyl, A denotes a divalent organic group of compounds,
n is a number from 0 to 200, preferably from 10 to 120, particularly preferably from 10 to 40,
and X⁻ is an anion.

The divalent group of compounds is preferably a C₁ to C₁₂ alkylene or alkoxyalkylene group, which can be substituted with one or more hydroxyl groups. The group —(CH₂)₃—O—CH₂—CH(OH)—CH₂— is particularly preferred.

The anion X⁻ can be a halide ion, an acetate, an organic carboxylate or a compound of the general formula RSO₃″, in which R has the meaning of C1 to C4 alkyl residues.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

in which A is the group —(CH₂)₃—O—CH₂—CH(OH)—CH₂—,
R is an alkyl residue having at least 8 C atoms and n is a number from 10 to 120.

Suitable silicone polymers having two terminal, quaternary ammonium groups are known under the INCI name Quaternium-80. These are dimethyl siloxanes having two terminal trialkylammonium groups. Such diquaternary polydimethyl siloxanes are sold by Evonik under the trade names Abil® Quat 3270, 3272 and 3474.

Cosmetic or dermatological preparations that are preferred according to the invention have the characterizing feature that they contain, relative to their weight, 0.01 to 10 wt. %, preferably 0.01 to 8 wt. %, particularly preferably 0.1 to 7.5 wt. % and in particular 0.2 to 5 wt. % of amino-functional silicone(s) and/or diquaternary silicone.

The compositions according to the invention can contain at least one polyammonium-polysiloxane compound as the silicone. The polyammonium-polysiloxane compounds can be purchased for example from GE Bayer Silicones under the trade name Baysilone®. The products with the names Baysilone TP 3911, SME 253 and SFE 839 are preferred here. The use of Baysilone TP 3911 as the active component of the compositions according to the invention is most particularly preferred. The polyammonium-polysiloxane compounds are used in the compositions according to the invention in an amount from 0.01 to 10 wt. %, preferably 0.01 to 7.5, particularly preferably 0.01 to 5.0 wt. %, most particularly preferably from 0.05 to 2.5 wt. %, relative in each case to the complete composition.

The cyclic dimethicones referred to under INCI as cyclomethicones can also be used to advantage according to the invention. Cosmetic or dermatological preparations are preferred here according to the invention that contain at least one silicone of formula (Si-4),

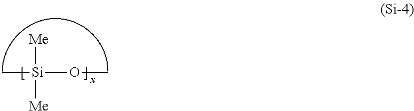

in which x denotes a number from 3 to 200, preferably from 3 to 10, more preferably from 3 to 7 and in particular 3, 4, 5 or 6.

Agents that are likewise preferred according to the invention have the characterizing feature that they contain at least one silicone of the formula (Si-5)

R₃Si—[O—SiR₂]ₓ—(CH₂)ₙ—[O—SiR₂]_{y}—O—SiR₃ (Si-5),

in which R denotes identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As further silicones in addition to the dimethicones, dimethiconols, amodimethicones and/or cyclomethicones according to the invention, water-soluble silicones can also be included in the compositions according to the invention.

Corresponding hydrophilic silicones are selected for example from the compounds of formulae (Si-6) and/or (Si-7). Silicone-based water-soluble surfactants that are preferred in particular are selected from the group of dimethicone copolyols, which are preferably alkoxylated, in particular polyethoxylated or polypropoxylated.

According to the invention dimethicone copolyols are understood to be preferably polyoxyalkylene-modified dimethyl polysiloxanes of the general formulae (Si-6) or (Si-7):

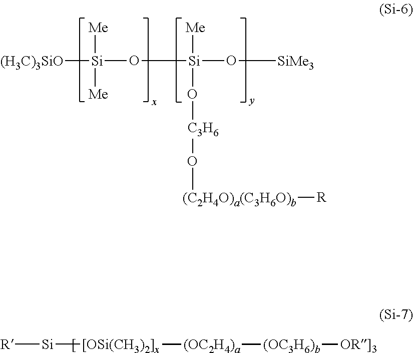

in which the residue R denotes a hydrogen atom, an alkyl group having 1 to 12 C atoms, an alkoxy group having 1 to 12 C atoms or a hydroxyl group, the residues R′ and R″ denote alkyl groups having 1 to 12 C atoms, x denotes a whole number from 1 to 100, preferably from 20 to 30, y denotes a whole number from 1 to 20, preferably 2 to 10, and a and b denote whole numbers from 0 to 50, preferably from 10 to 30.

Particularly preferred dimethicone copolyols within the meaning of the invention are for example the products sold commercially under the trade name SILWET (Union Carbide Corporation) and DOW CORNING. Dimethicone copolyols that are particularly preferred according to the invention are Dow Corning 190 and Dow Corning 193.

The dimethicone copolyols are contained in the compositions according to the invention in amounts from 0.01 to 10 wt. %, preferably 0.01 to 8 wt. %, particularly preferably 0.1 to 7.5 wt. % and in particular 0.1 to 5 wt. % of dimethicone copolyol, relative to the composition.

Finally, the silicone compounds are understood to include dimethiconols (Si8). The dimethiconols according to the invention can be both linear and branched and also cyclic or cyclic and branched. Linear dimethiconols can be represented by the following structural formula (Si8-I):

(SiOHR¹₂)—O—(SiR²₂—O—)ₓ—(SiOHR¹₂) (Si8-1)

Branched dimethiconols can be represented by the structural formula (Si8-II):

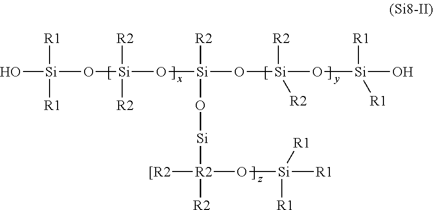

Residues R¹ and R² independently of each other denote hydrogen, a methyl residue, a C2 to C30 linear, saturated or unsaturated hydrocarbon residue, a phenyl residue and/or an aryl residue. The numbers x, y and z are whole numbers and each run independently of one another from 0 to 50,000. The molecular weights of the dimethiconols are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs.

The following commercial products are cited as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (these last four all GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (these last all Wacker-Chemie GmbH).

The dimethiconols (Si8) are contained in the compositions according to the invention in amounts from 0.01 to 10 wt. %, preferably 0.01 to 8 wt. %, particularly preferably 0.1 to 7.5 wt. % and in particular 0.1 to 5 wt. % of dimethiconol, relative to the composition.

Furthermore, in a most highly preferred embodiment polymers are included to increase the effect of the active ingredient complex (A). The cationic polymers can be homo- or copolymers, wherein the quaternary nitrogen groups are contained either in the polymer chain or preferably as a substituent at one or more of the monomers. The ammonium group-containing monomers can be copolymerized with non-cationic monomers. Suitable cationic monomers are unsaturated, radically polymerizable compounds bearing at least one cationic group, in particular ammonium-substituted vinyl monomers such as for example trialkyl methacryloxy alkylammonium, trialkyl acryloxy alkylammonium, dialkyl diallyl ammonium and quaternary vinyl ammonium monomers with cyclic groups containing cationic nitrogens, such as pyridinium, imidazolium or quaternary pyrrolidones, e.g. alkyl vinyl imidazolium, alkyl vinyl pyridinium, or alkyl vinyl pyrrolidone salts. The alkyl groups of these monomers are preferably low alkyl groups such as for example C1 to C7 alkyl groups, particularly preferably C1 to C3 alkyl groups.

The ammonium group-containing monomers can be copolymerized with non-cationic monomers. Suitable comonomers are for example acrylamide, methacrylamide, alkyl and dialkyl acrylamide, alkyl and dialkyl methacrylamide, alkyl acrylate, alkyl methacrylate, vinyl caprolactone, vinyl caprolactam, vinyl pyrrolidone, vinyl esters, for example vinyl acetate, vinyl alcohol, propylene glycol or ethylene glycol, the alkyl groups of these monomers preferably being C1 to C7 alkyl groups, particularly preferably C1 to C3 alkyl groups.

Suitable polymers having quaternary amine groups are for example the polymers described in the CTFA Cosmetic Ingredient Dictionary under the name Polyquaternium, such as methylvinyl imidazolium chloride/vinyl pyrrolidone copolymer (Polyquaternium-16) or quaternized vinyl pyrrolidone/dimethyl aminoethyl methacrylate copolymer (Polyquaternium-11).

Of the cationic polymers that can be included in the agent according to the invention, vinyl pyrrolidone/dimethylaminoethyl methacrylate methosulfate copolymer, which is sold under the commercial names Gafquat® 755 N and Gafquat® 734 by Gaf Co., USA, is suitable, of which Gafquat® 734 is particularly preferred. Further cationic polymers are for example the copolymer of polyvinyl pyrrolidone and imidazolimine methochloride, which is sold by BASF, Germany under the trade name Luviquat® HM 550, the terpolymer of dimethyldiallyl ammonium chloride, sodium acrylate and acrylamide, which is sold by Calgon/USA under the trade name Merquat® Plus 3300, and the vinyl pyrrolidone/methacrylamidopropyl trimethylammonium chloride copolymer sold by ISP under the trade name Gafquat® HS 100.

Homopolymers of the general formula (P1),

—{CH₂—[CR¹COO—(CH₂)ₘN⁺R²R³R⁴]}ₙX⁻

in which R¹=—H or —CH₃, R², R³ and R⁴ are selected independently of one another from C₁₋₄ alkyl, alkenyl or hydroxyalkyl groups, m=1, 2, 3 or 4, n is a natural number and X⁻ is a physiologically tolerable organic or inorganic anion. In the context of these polymers those for which at least one of the following conditions applies are preferred according to the invention: R¹ denotes a methyl group, R², R³ and R⁴ denote methyl groups, m has the value 2.

Suitable physiologically tolerable counterions X− are for example halide ions, sulfate ions, phosphate ions, methosulfate ions as well as organic ions such as lactate, citrate, tartrate and acetate ions. Halide ions, in particular chloride, are preferred.

A particularly suitable homopolymer is the optionally crosslinked poly(methacryloyloxyethyl trimethyl ammonium chloride) with the INCI name Polyquaternium-37. Such products are commercially available for example under the names Rheocare® CTH (Cosmetic Rheologies) and Synthalen® CR (3V Sigma).

The homopolymer is preferably used in the form of a non-aqueous polymer dispersion. Such polymer dispersions are commercially available under the names Salcare® SC 95 and Salcare® SC 96.

A copolymer that is preferred according to the invention is the crosslinked acrylamide-methacryloyloxyethyl trimethylammonium chloride copolymer. Such copolymers are commercially available under the name Salcare® SC 92.

Suitable cationic polymers that are derived from natural polymers are cationic derivatives of polysaccharides, for example cationic derivatives of cellulose, starch or guar. Also suitable are chitosan and chitosan derivatives. Cationic polysaccharides have the general formula

G-O—B—N+RₐR_{b}R_{c}X⁻ (P-3)

G is an anhydroglucose residue, for example starch or cellulose anhydroglucose;
B is a divalent group of compounds, for example alkylene, oxyalkylene, polyoxyalkylene or hydroxyalkylene;
Rₐ, R_{b} and R_{c} are independently of one another alkyl, aryl, alkylaryl, arylalkyl, alkoxyalkyl or alkoxyaryl, each having up to 18 C atoms, the total number of C atoms in Rₐ, R_{b} and Rₐ preferably being a maximum of 20;
X⁻ is a conventional counterion and is preferably chloride.

A cationic cellulose is sold under the name Polymer JR® 400 by Amerchol and has the INCI name Polyquaternium-10. A further cationic cellulose has the INCI name Polyquaternium-24 and is sold under the trade name Polymer LM-200 by Amerchol. Further commercial products are the compounds Celquat® H 100, Celquat® and L 200. The cited commercial products are preferred cationic celluloses.

Suitable cationic guar derivatives are sold under the trade name Jaguar and have the INCI name Guar Hydroxypropyltrimonium Chloride. Particularly suitable cationic guar derivatives are also sold furthermore by Hercules under the name N-Hance®. Further cationic guar derivatives are sold by Cognis under the name Cosmedia®. A preferred cationic guar derivative is the commercial product AquaCat® from Hercules. This raw material is a pre-dissolved cationic guar derivative.

A suitable chitosan is sold for example by Kyowa Oil & Fat, Japan, under the trade name Flonac®. A preferred chitosan salt is chitosonium pyrrolidone carboxylate, which is sold for example under the name Kytamer® PC by Amerchol, USA. Further chitosan derivatives are freely available commercially under the trade names Hydagen® CMF, Hydagen® HCMF and Chitolam® NB/101.

Other preferred cationic polymers are for example

cationic alkyl polyglycosides,
cationized honey, for example the commercial product Honeyquat® 50,
polymeric dimethyl diallyl ammonium salts and copolymers thereof with esters and amides of acrylic acid and methacrylic acid. The products available commercially under the names Merquat 100 (poly(dimethyl diallyl ammonium chloride)) and Merquat 550 (dimethyl diallyl ammonium chloride acrylamide copolymer) are examples of such cationic polymers,
vinyl pyrrolidone-vinyl imidazolium methochloride copolymers, such as are sold under the names Luviquat® FC 370, FC 550, FC 905 and HM 552,
quaternized polyvinyl alcohol,
and the polymers with quaternary nitrogen atoms in the polymer main chain known under the names Polyquaternium 2, Polyquaternium 17, Polyquaternium 18 and Polyquaternium 27, vinyl pyrrolidone-vinyl caprolactam-acrylate terpolymers, such as are offered commercially with acrylic acid esters and acrylic acid amides as the third monomer unit under the name Aquaflex® SF 40, for example.

Likewise suitable for use according to the invention are the copolymers of vinyl pyrrolidone, such as are available as the commercial products Copolymer 845 (manufacturer: ISP), Gaffix® VC 713 (manufacturer: ISP), Gafquat ASCP 1011, Gafquat®HS 110, Luviquat®8155 and Luviquat® MS 370.

The cationic polymers also include cationized protein hydrolysates, wherein the underlying protein hydrolysate can derive from animal sources, for example from collagen, milk or keratin, from plant sources, for example from wheat, corn, rice, potatoes, soy or almonds, from marine life forms, for example from fish collagen or algae, or from protein hydrolysates obtained by biotechnology. Typical examples of the cationic protein hydrolysates and derivatives according to the invention are the products that are listed under the INCI names in the “International Cosmetic Ingredient Dictionary and Handbook”, (seventh edition 1997, The Cosmetic, Toiletry, and Fragrance Association, 1101 17th Street, N.W., Suite 300, Washington, D.C. 20036-4702) and that are available commercially.

The cationic polymers are contained in the compositions according to the invention preferably in amounts from 0.01 to 10 wt. %, relative to the complete agent. Amounts from 0.05 to 5 wt. % are particularly preferred.

Amphoteric polymers can also be used as polymers. The term amphoteric polymers encompasses both polymers which contain both free amino groups and free —COOH or SO₃H groups in the molecule and which are capable of forming internal salts, and zwitterionic polymers which contain quaternary ammonium groups and —COO⁻ or SO₃⁻ groups in the molecule, and such polymers containing —COOH or SO₃H groups and quaternary ammonium groups.

Amphoteric and/or cationic polymers that are preferred according to the invention are polymers in which a cationic group is derived from at least one of the following monomers:

(i) monomers having quaternary ammonium groups of the general formula (Mono 1),

R¹—CH═CR²—CO—Z—(CₙH₂ₙ)—N⁽⁺⁾R²R³R⁴A⁽⁻⁾ (Mono1)

in which R¹ and R² independently of each other denote hydrogen or a methyl group and R³, R⁴ and R⁵ independently of one another denote alkyl groups having 1 to 4 carbon atoms, Z is an NH group or an oxygen atom, n is a whole number from 2 to 5 and A⁽⁻⁾ is the anion of an organic or inorganic acid,
(ii) monomers having quaternary ammonium groups of the general formula (Mono2),

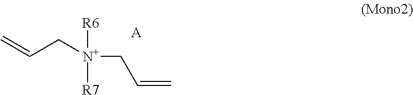

in which R⁶ and R⁷ independently of each other denote a (C₁ to C₄) alkyl group, in particular a methyl group, and
A⁻ is the anion of an organic or inorganic acid,
(iii) monomeric carboxylic acids of the general formula (Mono3),

R⁸—CH═CR⁹—COOH (Mono3)

in which R⁸ and R⁹ are independently of each other hydrogen or methyl groups.

Polymers in which monomers of type (i) are used, in which R³, R⁴ and R⁵ are methyl groups, Z is an NH group and A⁽⁻⁾ is a halide, methoxy sulfate or ethoxy sulfate ion, are most particularly preferred; acrylamidopropyl trimethylammonium chloride is a particularly preferred monomer (i). Acrylic acid is preferably used as the monomer (ii) for the cited polymers.

Particularly preferred amphoteric polymers are copolymers of at least one monomer (Mono1) or (Mono2) with the monomer (Mono3), in particular copolymers of monomers (Mono2) and (Mono3). Amphoteric polymers that are most particularly preferably used according to the invention are copolymers of diallyl dimethyl ammonium chloride and acrylic acid. These copolymers are sold under the INCI name Polyquaternium-22, inter alia under the trade name Merquat® 280 (Nalco).

In addition to a monomer (Mono1) or (Mono2) and a monomer (Mono3), the amphoteric polymers according to the invention can moreover additionally contain a monomer (Mono4)

(iv) monomeric carboxylic acid amides of the general formula (Mono4),

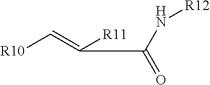

in which R¹⁰ and R¹¹ are independently of each other hydrogen or methyl groups and R¹² denotes a hydrogen atom or a (C₁ to C₈) alkyl group.

Amphoteric polymers based on a comonomer (Mono4) that are most particularly preferably used according to the invention are terpolymers of diallyldimethylammonium chloride, acrylamide and acrylic acid. These copolymers are sold under the INCI name Polyquaternium-39, inter alia under the trade name Merquat® Plus 3330 (Nalco).

The amphoteric polymers can generally be used both directly and in the form of the salt, which is obtained by neutralization of the polymers, with an alkali hydroxide for example.

The amphoteric polymers are contained in the agents according to the invention preferably in amounts from 0.01 to 10 wt. %, relative to the complete agent. Amounts from 0.01 to 5 wt. % are particularly preferred.

The anionic polymers are anionic polymers having carboxylate and/or sulfonate groups. Examples of anionic monomers which can constitute such polymers are acrylic acid, methacrylic acid, crotonic acid, maleic anhydride and 2-acrylamido-2-methylpropane sulfonic acid. Some or all of the acid groups therein can be present as the sodium, potassium, ammonium, mono- or triethanolammonium salt. Preferred monomers are 2-acrylamido-2-methylpropane sulfonic acid and acrylic acid. Anionic polymers containing as the sole monomer or as a co-monomer 2-acrylamido-2-methylpropane sulfonic acid, in which some or all of the sulfonic acid group can be present as the sodium, potassium, ammonium, mono- or triethanolammonium salt, have proved to be most particularly effective.

The homopolymer of 2-acrylamido-2-methylpropane sulfonic acid, which is available commercially for example under the name Rheothik®11-80, is particularly preferred.

Within this embodiment it can be preferable to use copolymers consisting of at least one anionic monomer and at least one non-ionogenic monomer. Reference is made to the aforementioned substances with regard to the anionic monomers. Preferred non-ionogenic monomers are acrylamide, methacrylamide, acrylic acid ester, methacrylic acid ester, vinyl pyrrolidone, vinyl ether and vinyl ester.

Preferred anionic copolymers are acrylic acid-acrylamide copolymers as well as in particular polyacrylamide copolymers with monomers containing sulfonic acid groups. Such a polymer is contained in the commercial product Sepigel®305 from SEPPIC.

The sodium acryloyldimethyltaurate copolymers sold under the name Simulgel®600 as a compound with isohexadecane and Polysorbate-80 have also proved to be particularly effective according to the invention.

Likewise preferred anionic homopolymers are uncrosslinked and crosslinked polyacrylic acids. Allyl ethers of pentaerythritol, of sucrose and of propylene can be preferred crosslinking agents here. Such compounds are available commercially for example under the trademark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is available commercially under the name Stabileze® QM.

The anionic polymers are contained in the agents according to the invention preferably in amounts from 0.05 to 10 wt. %, relative to the complete agent. Amounts from 0.1 to 5 wt. % are particularly preferred.

A polyurethane that is most particularly preferred according to the invention is sold under the trade name Luviset® PUR (BASF).

In a further embodiment the agents according to the invention can contain non-ionogenic polymers.

Suitable non-ionogenic polymers are for example:

vinyl pyrrolidone/vinyl ester copolymers, such as are sold for example under the trademark Luviskol® (BASF). Luviskol® VA 64 and Luviskol® VA 73, both of which are vinyl pyrrolidone/vinyl acetate copolymers, are likewise preferred non-ionic polymers; cellulose ethers, such as hydroxypropyl cellulose, hydroxyethyl cellulose and methylhydroxypropyl cellulose, such as are sold for example under the trademarks Culminal® and Benecel® (AQUALON), and Natrosol® types (Hercules);
starch and derivatives thereof, in particular starch ethers, for example Structure® XL (National Starch), a multifunctional, salt-tolerant starch;
shellac;
polyvinyl pyrrolidones, such as are sold for example under the name Luviskol® (BASF).

The non-ionic polymers are contained in the compositions according to the invention preferably in amounts from 0.05 to 10 wt. %, relative to the complete agent. Amounts from 0.1 to 5 wt. % are particularly preferred.

It is also possible according to the invention for the preparations used to contain a plurality of, in particular two different polymers of the same charge and/or an ionic and an amphoteric and/or non-ionic polymer.

The polymers (P) are contained in the compositions used according to the invention preferably in amounts from 0.01 to 30 wt. %, relative to the complete composition. Amounts from 0.01 to 25, in particular from 0.01 to 5 wt. %, are particularly preferred.

The compositions according to the invention can of course contain further ingredients conventionally used in cosmetic compositions.

Emulsifiers that can be used according to the invention are for example
addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group,
C₁₂-C₂₂ fatty acid monoesters and diesters of addition products of 1 to 30 mol of ethylene oxide with polyols having 3 to 6 carbon atoms, in particular with glycerol,
ethylene oxide and polyglycerol addition products with methyl glucoside fatty acid esters, fatty acid alkanol amides and fatty acid glucamides,
C₈-C₂₂ alkyl mono- and oligoglycosides and ethoxylated analogs thereof, wherein degrees of oligomerization of 1.1 to 5, in particular 1.2 to 2.0, and glucose as the sugar component are preferred,
mixtures of alkyl (oligo)glucosides and fatty alcohols, for example the commercially available product Montanov® 68,
addition products of 5 to 60 mol of ethylene oxide with castor oil and hydrogenated castor oil,
partial esters of polyols having 3 to 6 carbon atoms with saturated fatty acids having 8 to 22 C atoms,
sterols, from both animal tissue (zoosterols, cholesterol, lanosterol) and vegetable fats (phytosterols, ergosterol, stigmasterol, sitosterol) or from fungi and yeasts (mycosterols),
phospholipids (lecithins, phosphatidyl cholines),
fatty acid esters of sugars and sugar alcohols such as sorbitol,
polyglycerols and polyglycerol derivatives such as for example polyglycerol poly-12-hydroxystearate (commercial product Dehymuls® PGPH).

The agents according to the invention contain the emulsifiers preferably in amounts from 0.1 to 25 wt. %, in particular 0.5 to 15 wt. %, relative to the complete agent.

Linear and/or branched, saturated and/or unsaturated fatty acids having 6 to 30 carbon atoms can be used as fatty acids (Fatac). Fatty acids having 10 to 22 carbon atoms are preferred. Examples which can be cited include the isostearic acids, such as the commercial products Emersol® 871 and Emersol® 875, and isopalmitic acids such as the commercial product Edenor® IP 95, as well as all other fatty acids sold under the Edenor® trade names (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof. The fatty acid cuts obtainable from coconut oil or palm oil are conventionally particularly preferred; as a rule the use of stearic acid is preferred in particular.

The amount used here is 0.1 to 15 wt. %, relative to the complete agent. The amount is preferably 0.5 to 10 wt. %, wherein amounts of 1 to 5 wt. % can be most particularly advantageous.

Saturated, mono- or polyunsaturated, branched or unbranched fatty alcohols having C₆ to C₃₀, preferably C₁₀ to C₂₂ and most particularly preferably C₁₂ to C₂₂ carbon atoms can be used as fatty alcohols (Fatal). Suitable for use within the meaning of the invention are for example decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as the Guerbet alcohols thereof, wherein this list is intended to be of an exemplary and non-limiting nature. However, the fatty alcohols derive from preferably natural fatty acids, wherein it can conventionally be assumed that they are obtained from the esters of fatty acids by reduction. Likewise suitable for use according to the invention are fatty alcohol cuts constituting a mixture of different fatty alcohols. Such substances are available commercially for example under the names Stenol®, e.g. Stenol® 1618 or Lanette®, e.g. Lanette® 0 or Lorol®, e.g. Lorol® C8, Lorol® C14, Lorol® C18, Lorol® C₈₋₁₈, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols can of course also be used according to the invention, such as are available commercially for example under the names Corona®, White Swan®, Coronet® or Fluilan®. The fatty alcohols are used in amounts from 0.1 to 30 wt. %, relative to the complete preparation, preferably in amounts from 0.1 to 20 wt. %.

Solid paraffins or isoparaffins, carnauba waxes, beeswaxes, candelilla waxes, ozokerites, ceresin, spermaceti wax, sunflower wax, fruit waxes such as for example apple wax or citrus wax, PE or PP microwaxes can be used according to the invention as natural or synthetic waxes (Fatwax). Such waxes are available for example via Kahl & Co., Trittau.

The amount used is 0.1 to 50 wt. %, relative to the complete agent, preferably 0.1 to 20 wt. % and particularly preferably 0.1 to 15 wt. %, relative to the complete agent.

The total amount of oil and fat components in the agents according to the invention is conventionally 0.5 to 75 wt. %, relative to the complete agent. Amounts from 0.5 to 35 wt. % are preferred according to the invention.

Protein hydrolysates and/or derivatives thereof (P) are a further synergistic active ingredient according to the invention in the compositions according to the invention with the active ingredient complex according to the invention.

According to the invention protein hydrolysates of both plant and animal or marine or synthetic origin can be used.

Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Also preferred according to the invention are plant protein hydrolysates, such as for example soy, almond, pea, moringa, potato and wheat protein hydrolysates. Such products are available for example under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Serobiologiques.

Further protein hydrolysates that are preferred according to the invention are of marine origin. They include for example collagen hydrolysates of fish or algae and protein hydrolysates of mussels or pearl hydrolysates. Examples of pearl extracts according to the invention are the commercial products Pearl Protein Extract BG® or Crodarom® Pearl.

It is most highly preferable, however, for casein to be used as the protein hydrolysate. The use of casein in the compositions together with rambutan oil leads to tactile effects on the skin and hair that are clearly perceptible by sensory means. Owing to its tactile properties, hair treated therewith thus feels markedly stronger and more vigorous. The surface is smooth and soft, while at the same time the treatment of the surface with the active ingredient combination is still perceptible.

The protein hydrolysates (P) are contained in the compositions in concentrations from 0.001 wt. % to 20 wt. %, preferably from 0.05 wt. % to 5 wt. % and most particularly preferably in amounts from 0.05 wt. % to 5 wt. %.

The effect of the compositions according to the invention can be further increased by means of a 2-pyrrolidinone-5-carboxylic acid and derivatives thereof. The sodium, potassium, calcium, magnesium or ammonium salts are preferred, in which the ammonium ion bears one to three C₁ to C₄ alkyl groups in addition to hydrogen. The sodium salt is most particularly preferred. The amounts used in the agents according to the invention are 0.05 to 10 wt. %, relative to the complete agent, particularly preferably 0.1 to 5, and in particular 0.1 to 3 wt. %.

A further preferred group of ingredients of the compositions according to the invention with the active ingredient complex according to the invention are vitamins, provitamins or vitamin precursors.

Vitamins, provitamins and vitamin precursors are particularly preferred that are assigned to groups A, B, C, E, F and H.

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents according to the invention contain the vitamin A component preferably in amounts from 0.05 to 1 wt. %, relative to the complete preparation.

The vitamin B group or vitamin B complex includes inter alia:

vitamin B₁ (thiamine)
vitamin B₂ (riboflavin)
vitamin B₃. The compounds nicotinic acid and nicotinic acid amide (niacinamide) are often included under this term. Preferred according to the invention is nicotinic acid amide, which is preferably contained in the agents used according to the invention in amounts from 0.05 to 1 wt. %, relative to the complete agent
vitamin B₅ (pantothenic acid, panthenol and pantolactone). Within the context of this group panthenol and/or pantolactone is preferably used. Derivatives of panthenol which can be used according to the invention are in particular the esters and ethers of panthenol as well as cationically derivatized panthenols. Individual representatives are for example panthenol triacetate, panthenol monoethyl ether and the monoacetate thereof, and cationic panthenol derivatives. Pantothenic acid is preferably used in the present invention as a derivative in the form of the more stable calcium salts and sodium salts (Ca pantothenate, Na pantothenate)
vitamin B₆ (pyridoxine as well as pyridoxamine and pyridoxal).

The cited compounds of the vitamin B type, in particular vitamin B₃, B₅ and B₆, are contained in the agents according to the invention preferably in amounts from 0.05 to 10 wt. %, relative to the complete agent. Amounts from 0.1 to 5 wt. % are particularly preferred.

Vitamin C (ascorbic acid). Vitamin C is used in the agents according to the invention preferably in amounts from 0.1 to 3 wt. %, relative to the complete agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular the esters such as acetate, nicotinate, phosphate and succinate, are preferably contained in the agents according to the invention in amounts from 0.05 to 1 wt. %, relative to the complete agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably contained in the agents according to the invention in amounts from 0.0001 to 1.0 wt. %, in particular in amounts from 0.001 to 0.01 wt. %.

The compositions according to the invention preferably contain vitamins, provitamins and vitamin precursors from groups A, B, E and H. Panthenol, pantolactone, pyridoxine and derivatives thereof as well as nicotinic acid amide and biotin are particularly preferred.

A particularly preferred group of ingredients in the cosmetic compositions according to the invention is constituted by the betaines listed below: carnitine, carnitine tartrate, carnitine magnesium citrate, acetyl carnitine, betalains, 1,1-dimethyl proline, choline, choline chloride, choline bitartrate, choline dihydrogen citrate and the compound N,N,N-trimethylglycine, which is classed in the literature as betaine.

Carnitine, histidine, choline and betaine are preferably used. In a particularly preferred embodiment of the invention L-carnitine tartrate is used as the active ingredient.

A particularly substantial ingredient is taurine and/or a taurine derivative. Taurine is understood to be exclusively 2-aminoethane sulfonic acid, and a derivative to be the explicitly cited taurine derivatives. The taurine derivatives are understood to be N-monomethyl taurine, N,N-dimethyltaurine, taurine lysylate, taurine tartrate, taurine omithate, lysyl taurine and ornithyl taurine. Further taurine derivatives within the meaning of the present invention are taurocholic acid and hypotaurine.

Agents according to the invention which, relative to their weight, contain 0.0001 to 10.0 wt. %, preferably 0.0005 to 5.0 wt. %, particularly preferably 0.001 to 2.0 wt. % and in particular 0.001 to 1.0 wt. % of taurine and/or a taurine derivative are particularly preferred.

In a further embodiment that is preferred according to the invention the compositions according to the invention contain bioquinones. In the agents according to the invention suitable bioquinones are understood to be one or more ubiquinones and/or plastoquinones. The ubiquinones that are preferred according to the invention have the following formula:

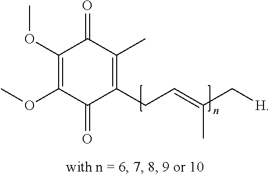

Coenzyme Q-10 is most preferred here.

Preferred compositions according to the invention contain purine and/or purine derivatives in narrower quantity ranges. Cosmetic agents that are preferred according to the invention have the characterizing feature that they contain, relative to their weight, 0.001 to 2.5 wt. %, preferably 0.0025 to 1 wt. %, particularly preferably 0.005 to 0.5 wt. % and in particular 0.01 to 0.1 wt. % of purine(s) and/or purine derivative(s). Cosmetic agents that are preferred according to the invention have the characterizing feature that they contain purine, adenine, guanine, uric acid, hypoxanthine, 6-purinethiol, 6-thioguanine, xanthine, caffeine, theobromine or theophylline In hair cosmetics preparations caffeine is most preferred.

In a further preferred embodiment of the present invention the cosmetic agent contains ectoine ((S)-2-methyl-1,4,5,6-tetrahydro-4-pyrimidine carboxylic acid).

Particularly preferred according to the invention are agents which, relative to their weight, contain 0.00001 to 10.0 wt. %, preferably 0.0001 to 5.0 wt. % and in particular 0.001 to 3 wt. % of active ingredients from the group formed from carnitine, taurine, coenzyme Q-10, ectoine, a vitamin of the B series, a purine and derivatives thereof or physiologically tolerable salts.

In a further embodiment the agents according to the invention should additionally contain at least one UV light screening filter. UVB filters can be oil-soluble or water-soluble.

Examples of oil-soluble substances that can be cited include:
3-benzylidene camphor, for example 3-(4-methylbenzylidene)camphor;
4-aminobenzoic acid derivatives, preferably 4-(dimethylamino)benzoic acid-2-ethylhexyl ester, 4-(dimethylamino)benzoic acid-2-octyl ester and 4-(dimethylamino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3-phenylcinnamic acid-2-ethylhexyl ester (octocrylene);
esters of salicylic acid, preferably salicylic acid-2-ethylhexyl ester, salicylic acid-4-isopropylbenzyl ester, salicylic acid homomethyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzomalonic acid di-2-ethylhexyl ester;
triazine derivatives, such as for example 2,4,6-trianilino-(p-carbo-2′-ethyl-1′-hexyloxy)-1,3,5-triazine and octyl triazone.
propane-1,3-diones, such as for example 1-(4-tert-butylphenyl)-3-(4′-methoxyphenyl)propane-1,3-dione.

Suitable water-soluble substances include:
2-phenylbenzimidazole-5-sulfonic acid and alkali, alkaline-earth, ammonium, alkyl ammonium, alkanol ammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as for example 4-(2-oxo-3-bornylidene methyl)benzenesulfonic acid and 2-methyl-5-(2-oxo-3-bornylidene)sulfonic acid and salts thereof.

As typical UV-A filters, derivatives of benzoyl methane are suitable in particular, such as for example 1-(4′-tert-butylphenyl)-3-(4′-methoxyphenyl)propane-1,3-dione or 1-phenyl-3-(4′-isopropylphenyl)propane-1,3-dione. The UV-A and UV-B filters can naturally also be used in mixtures. In addition to the cited soluble substances, insoluble pigments, in particular finely dispersed metal oxides or salts, are suitable for this purpose, such as for example titanium dioxide, zinc oxide, iron oxide, aluminum oxide, cerium oxide, zirconium oxide, silicates (talc), barium sulfate and zinc stearate. The particles should have an average diameter of less than 100 nm, preferably between 5 and 50 nm and in particular between 15 and 30 nm. They can have a spherical form, but such particles having an ellipsoid form or other form deviating from the spherical shape can also be used.

Finally, further advantages arise through the use of plant extracts (L) in the compositions according to the invention. Preferred above all according to the invention are the extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, valerian, lady's smock, wild thyme, yarrow, thyme, melissa, restharrow, coltsfoot, marshmallow, meristem, ginseng, coffee, cocoa, moring a, ginger root and ayurvedic plant extracts such as for example Aegle mannelos (bilva), Cyperus rotundus (nagarmotha), Emblica officinalis (amalaki), Morinda citrifolia (ashyuka), Tinospora cordifolia (guduchi), Santalum album (chandana), Crocus sativus (kumkuma), Cinnamonum zeylanicum and Nelumbo nucifera (kamala), grasses such as wheat, barley, rye, oats, spelt, corn, the various types of millet (proso millet, finger millet, foxtail millet as examples), sugar cane, ryegrass, meadow foxtail, false oat-grass, bentgrass, meadow fescue, moor grass, bamboo, cottongrass, pennisetums, Andropogonodeae (Imperata cylindrical, also known as blood grass or cogon grass), buffalo grass, cord grass, dog's tooth grass, lovegrass, Cymbopogon (citronella grass), Oryzeae (rice), Zizania (wild rice), marram grass, blue oatgrass, soft-grass, quaking grasses, speargrass, couch grass and Echinacea, in particular Echinacea angustifolia DC, Echinacea paradoxa (Norton), Echinacea simulata, E. atrorubens, E. tennesiensis, Echinacea strigosa (McGregor), Echinacea laevigata, Echinacea purpurea (L.) Moench and Echinacea pallida (Nutt), all types of vine and pericarp of Litchi chinensis.

The plant extracts can be used according to the invention in both pure and diluted form. If they are used in diluted form they conventionally contain approximately 2 to 80 wt. % of active substance and as the solvent the extracting agent or mixture of extracting agents used to obtain them.

The cosmetic agents can furthermore contain additional active ingredients, auxiliary substances and additives, such as for example
texturizing agents such as maleic acid and lactic acid,
swelling agents such as urea, allantoin, carbonates or hydantoin,
dimethyl isosorbide and cyclodextrins,
dyes to color the agent,
anti-dandruff active ingredients such as piroctone olamine, zinc omadine and climbazole,
complexing agents such as EDTA, NTA, β-alanine diacetic acid and phosphonic acids,
opacifiers such as latex, styrene/PVP and styrene/acrylamide copolymers,
pearlescent agents such as ethylene glycol mono- and distearate as well as PEG-3 distearate,
pigments,
stabilizing agents for hydrogen peroxide and other oxidizing agents,
propellants such as propane-butane mixtures, N₂O, dimethyl ether, CO₂ and air,
antioxidants,
perfume oils, scents and fragrances.

With regard to further optional components and to the amounts of these components used, reference is expressly made to the relevant manuals known to the person skilled in the art.

The invention also provides

The present invention secondly provides the use of the composition according to the invention for the restructuring of keratinic fibers, in particular human hair.

The present invention thirdly provides the use of the composition according to the invention for the restructuring and deep-down care of keratinic fibers inside the fibers, in particular human hair.

The present invention fourthly provides a method for treating keratinic fibers, in particular human hair, in which a composition according to the invention is applied to the keratinic fibers and after a contact period is rinsed out of the keratinic fibers again. The contact period is preferably a few seconds to 100 minutes, particularly preferably 1 to 50 minutes and most particularly preferably 1 to 30 minutes.

The present invention fifthly provides a method for treating keratinic fibers, in particular human hair, in which a composition according to the invention is applied to the keratinic fibers and remains there until the next time the keratinic fibers, in particular human hair, are washed.

In both of the aforementioned methods the keratinic fibers can be wet or dry. It is preferable in both of the aforementioned methods for the composition according to the invention to be applied to damp, towel-dry keratinic fibers. The keratinic fibers treated by the aforementioned methods can naturally be subjected to further treatments in the usual way, such as for example blow-drying, styling, combing or brushing.

### Examples and proof of effectiveness

Unless otherwise specified, all stated amounts are parts by weight.

### 1. Proof of Effectiveness of Restructuring

Pretreatment

Strands from Alkinco (1.0 g, code 6634) were shampooed with 2.0 g of a solution of 10.0 wt. % sodium lauryl ether sulfate (2EO) for 2 minutes. Then the hair strand was rinsed for 1 minute under running water at 23° C. and dried with a hair dryer. This was followed by a conventional permanent wave treatment with 2.0 g of the commercial product Poly Lock-Normale Dauerwelle. In this permanent wave treatment the fibers were exposed in a first step to the reducing solution (containing 7.9 wt. % of thioglycolic acid) for 40 minutes at room temperature, rinsed for 1 minute under running water with pure water at a temperature of 23° C., dried with a towel and then fixed at room temperature for 10 minutes with 2.0 g of an oxidizing solution. The oxidizing solution contained 2.6 wt. % of hydrogen peroxide. After the oxidative treatment the fibers were again rinsed for 1 minute under running water at 23° C. and then dried with a hair dryer. The permanent wave treatment was performed twice in total under identical conditions.

Aftertreatment

The strands were each immersed for 30 minutes in an aqueous solution of the individual active ingredients with a pH of 7, which had been established with sodium hydroxide solution or hydrochloric acid, at a temperature of 32° C. Following treatment with the individual active ingredient composition, each strand of hair was rinsed for 1 minute with clean water, dried in air and allowed to rest for 16 hours.

Proof of the hair-structuring effect using HP-DSC

The melting points listed in Table 1 below were determined by DSC analysis (Perkin Elmer DSC-7). A precise description of the method can be found for example in DE 196 173 95 A1.

| Active ingredient or active ingredient complex | Melting point in ° C. |
|---|---|
| 1. Untreated hair (control) | 151.4 |
| 2. Cold-waved hair without active ingredient | 147.4 |
| 3. Pure water pH 3 | 149.5 |
| 4. 0.2% Dimethylsilanol hyaluronate and 0.2% | 151.4 |
| glycerol in water at pH 3 | |

The significance level of the active ingredient complex according to the invention shown in 4 as compared with the value for untreated hair is >99%.

### 2. Further examples

Unless otherwise specified, all stated amounts are parts by weight. The following formulations were prepared using known production methods.

Conditioning Shampoo:

| | wt. % |
|---|---|
| Texapon N 70 | 14.0 |
| Dehyton G | 7.0 |
| Salicylic acid | 0.2 |
| D-Panthenol (75%) | 0.5 |
| Na benzoate | 0.5 |
| Polyquaternium-10 | 1.5 |
| Citric acid, anhydrous | 0.5 |
| Tocopheryl acetate | 0.2 |
| Sodium chloride | 1.5 |
| Sodium hydroxide solution, 50% | 0.2 |
| PEG-40 hydrogenated castor oil | 0.5 |
| Hydrolyzed keratin | 0.1 |
| Rambutan oil | 4.0 |
| Perfume, preservatives | qs |
| Water | to 100 |

Conditioner:

| | wt. % |
|---|---|
| Paraffin oil, liquid | 1.0 |
| Dehyquart F75 | 1.5 |
| Coconut oil | 5.0 |
| Varisoft W 75 PG | 1.5 |
| Cetearyl alcohol | 4.0 |
| Eumulgin B2 | 0.4 |
| Propylparaben | 0.15 |
| Cutina CP | 0.7 |
| Stearamidopropyl dimethylamine | 1.0 |
| Dehyquart A-CA | 3.5 |
| Lactic acid | 0.5 |
| Methylparaben | 0.3 |
| Phenoxyethanol | 0.4 |
| Pantolactone | 0.5 |
| Gluadin W20 | 1.0 |
| Niacinamide | 0.2 |
| Perfume | 0.4 |
| Water | to 100 |

Leave-on Treatment:

| | wt. % |
|---|---|
| Synthalen K | 0.4 |
| Sepigel 305 | 1.5 |
| Dehyquart F 75 | 0.3 |
| Polymer JR 400 | 0.2 |
| Neutrol TE | 0.4 |
| Rambutan oil | 4.0 |
| Perfume | 0.4 |
| Tocopheryl acetate | 0.2 |
| Silk protein hydrolysate | 0.5 |
| PEG-40 hydrogenated castor oil | 0.5 |
| Dow Corning 1401 Fluid | 1.5 |
| Ethanol (96%) | 15.0 |
| Water | to 100 |

Intensive Mask:

| | wt. % |
|---|---|
| Varisoft W 75 PG | 3.5 |
| Paraffin oil, liquid | 1.0 |
| Dehyquart F 75 | 2.0 |
| Stearamidopropyl dimethylamine | 1.0 |
| Palm kernel oil | 5.0 |
| Cetearyl alcohol | 7.0 |
| Cutina GMS-V | 1.0 |
| Emulmetik 100 | 0.4 |
| Isopropyl myristate | 1.0 |
| Citric acid | 0.7 |
| Dehyquart A-CA | 3.0 |
| Salcare SC 96 | 0.5 |
| Perfume | 0.4 |
| D-Panthenol (75%) | 0.5 |
| Nutrilan Keratin W | 0.5 |
| Niacinamide | 0.2 |
| Methylparaben | 0.4 |
| Phenoxyethanol | 0.4 |
| Water | to 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for treating keratinic fibers, in particular human hair, comprising:
a) dimethylsilanol hyaluronate,
b) glycerol and
c) a cosmetic carrier.

2. The composition according to claim 1, wherein furthermore at least one further active ingredient, selected from the group formed from carnitine, taurine, coenzyme Q-10, ectoine, a vitamin from the B series or a purine, is included.

3. The composition according to claim 1, wherein at least one plant extract is included as a further active ingredient.

4. A method for treating keratinic fibers, in particular human hair, in which a composition according to claim 1 is applied to the keratinic fibers and after a contact period is rinsed out of the keratinic fibers again.

5. A method for treating keratinic fibers, in particular human hair, in which a composition according to claim 1 is applied to the keratinic fibers and remains there until the next time the keratinic fibers, in particular human hair, are washed.
